(19) United States
(12) Patent Application Publication
(10) Pub. No.: US 2025/0262340 A1
(43) Pub. Date: Aug. 21, 2025

(54) PROCESSING TRAY AND METHODS OF USE THEREOF

(71) Applicant: Think Surgical, Inc., Fremont, CA (US)

(72) Inventor: Timothy Pack, Fremont, CA (US)

(73) Assignee: Think Surgical, Inc., Fremont, CA (US)

(21) Appl. No.: 18/856,699

(22) PCT Filed: Apr. 17, 2023
(86) PCT No.: PCT/US2023/018841

§ 371 Date: Oct. 14, 2024

Related U.S. Application Data
(60) Provisional application No. 63/330,856, filed on Apr. 14, 2022.

(51) Int. Cl. A61L 2/26

(52) U.S. Cl. CPC A61L 2/26; A61L 2202/14; A61L 2202/24

(57) ABSTRACT

A processing tray for a device having a subassembly is provided that includes a base. One or more receiving appurtenances are positioned on the base or an insert supported by the base. The receiving appurtenances are complementary to the device for retaining the device at a predetermined location. One or more arresting appurtenances are present and adapted to prevent receipt of the device at the predetermined location if the subassembly is coupled with the device. The arresting appurtenances may contact the subassembly when the subassembly is coupled with the device to prevent such receipt. A method of processing a device includes decoupling a subassembly from an object to yield the device to be processed. The device is placed in contact at the predetermined location to prevent receipt of the device if the subassembly is still coupled to the device. As a result, accidental processing of the subassembly is prevented.

### RELATED APPLICATIONS

This application claims priority of U.S. Provisional Application Ser. No. 63/330,856, filed Apr. 14, 2022; the contents of which are hereby incorporated by reference.

### FIELD OF THE INVENTION

The invention in general relates to processing trays and in particular to methods of use thereof that protect process intolerant components of equipment being subjected to the process.

### BACKGROUND OF THE INVENTION

Devices routinely require process treatment before usage or re-usage. Such processes may include solution emersion for cleaning, greasing, electroplating, or sterilization; exposure to gases for deposition coating, reactive cleaning, or sterilization; and radiation exposure for sterilization. By way of example, devices such as tools, instruments, and implants used in medical procedures and non-medical processes (e.g., cleanroom environments, food processing/preparation) require cleaning, sterilization, and/or surface modification prior to use. Still other requirements might include the need to apply a surface coating or etch material from the device surface. Exposing a device to sterilization conditions such as dry heat, radiation, steam, or sterilizing liquids or gases (e.g., ethylene oxide (EtO), ozone, mixed oxides of nitrogen, chlorine dioxide) can effectively sterilize the device.

Often a device or collection of devices are placed in a processing tray to facilitate handling during the desired process. The tray affords representative functions such as facilitating loading, limiting device handling, organizing devices for shipping, processing, and use, or promoting robotic handling. Receiving appurtenances are readily provided to facilitate proper positioning of a device at a predetermined location on the process tray. The process tray with the device(s) therein is then amenable to being exposed to the process conditions, such as sterilization.

In some cases, it may be desirable to remove certain device components from a device prior to processing. Exemplary reasons to disassemble a device prior to processing include user preference, manufacturer preference, potential damage of a device component potentially induced by the processing conditions, a device component occlusion of a particular portion or part of the device that needs to be directly exposed to the processing conditions. By way of example, electrical components, such as batteries, are often damaged by processing conditions, while plastics may embrittle or lose dimensional integrity under metal processing conditions. Specifically, ethylene oxide is known to have damaging effects on electrical components during EtO sterilization. Unfortunately, user error can result in the failure to disassemble a device with susceptible components prior to processing.

Thus, there exists a need for a tray and a method of use thereof that ensure certain device components are removed from a device prior to exposing the tray to a given process.

### SUMMARY OF THE INVENTION

A processing tray for a device having a subassembly is provided that includes a base. One or more receiving appurtenances are positioned on the base or an insert supported by the base. The one or more receiving appurtenances complementary to the device for retaining the device at a predetermined location. One or more arresting appurtenances are present and adapted to prevent receipt of the device at the predetermined location if the subassembly is coupled with the device. The arresting appurtenances may contact the subassembly when the subassembly is coupled with the device to prevent such receipt.

A method of processing a device includes decoupling a subassembly from an object to yield the device to be processed. The device is placed in contact with a portion of the one or more receiving appurtenances for retaining the device at the predetermined location wherein the one or more arresting appurtenances are positioned in relation to the one or more receiving appurtenances to prevent receipt of the device if the subassembly were still coupled to the device. The device is then exposed to the processing condition. As a result, accidental processing of the subassembly is prevented.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a top view of an embodiment of an inventive processing tray containing disparate exemplary devices;

FIG. 2 is a perspective view of the tray of FIG. 1;

FIG. 3 is an exploded perspective view of the tray with a single device shown and decoupled from a subassembly;

FIG. 4 is a side view of a hand-held robotic device shown in FIG. 1;

FIG. 5 is a perspective view of an arresting appurtenance for preventing receipt of the hand-held robotic device of FIG. 1 in the tray when a subassembly is coupled to the hand-held robotic device;

FIG. 6A is a top view of the tray with the hand-held robotic device with receiving appurtenances and arresting appurtenances engaging the device;

FIG. 6B is a cross-sectional view of FIG. 6A along the line A-A′ showing the arresting appurtenances retaining a door of the hand-held robotic device after removal of a subassembly therefrom;

FIG. 7 is an exploded perspective view of an inventive tray adapted to retain two tracking arrays as the devices, along with an insert on which receiving appurtenances and arresting appurtenances are supported;

FIG. 8 is a magnified, exploded perspective view of the insert, and the devices, receiving appurtenances, and arresting appurtenances of FIG. 7;

FIGS. 9A and 9B depict a first tracking array device as a front view (FIG. 9A) and a rear view (FIG. 9B);

FIG. 10 is a perspective view of an arresting appurtenance for preventing receipt of tracking array devices with a subassembly therein;

FIG. 11 is a perspective view of the arresting appurtenance of FIG. 10 with the tracking array devices placed therein after decoupling of the subassembly therefrom;

FIG. 12 is an exploded perspective view of an embodiment of an inventive tray with a device adapted to be supported on an insert adapted to retain a receiving appurtenance and an arresting appurtenance;

FIG. 13 is an exploded perspective view of the insert, device, the receiving appurtenance and the arresting appurtenance of FIG. 12;

FIG. 14 is a side view of a digitizer device having an aperture in the digitizer handle;

FIG. 15 is a perspective view of an embodiment of an inventive arresting appurtenance;

FIG. 16 a perspective view of the device of FIG. 14 coupled to the arresting appurtenance of FIG. 15 when the subassembly is removed to clear the aperture;

FIG. 17 is a perspective view of an embodiment of a combined receiving and arresting appurtenance (CRAA); and

### DESCRIPTION OF THE INVENTION

The present invention has utility in general as a processing tray and in particular as a sterilization tray for sterilizing devices. The tray has one or more arresting appurtenances to prevent receipt of a device on the tray at a predetermined location when a subassembly is coupled thereto. This ensures the subassembly is not exposed to the processing conditions. Exemplary subassemblies that can be damaged by processing or prevent processing access to internal parts of an object defined by the combination of the device and the subassembly include electrical components and those that are damaged by chemical, thermal, or actinic exposures associated with the processing. Electrical components that are often susceptible to processing damage illustratively include at least one of a battery, a processor, a memory, a peripheral electronic device connection, an electrical connection, a circuit board, a transmitter, a modem, a capacitor, an electrical motor, or a combination thereof.

The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

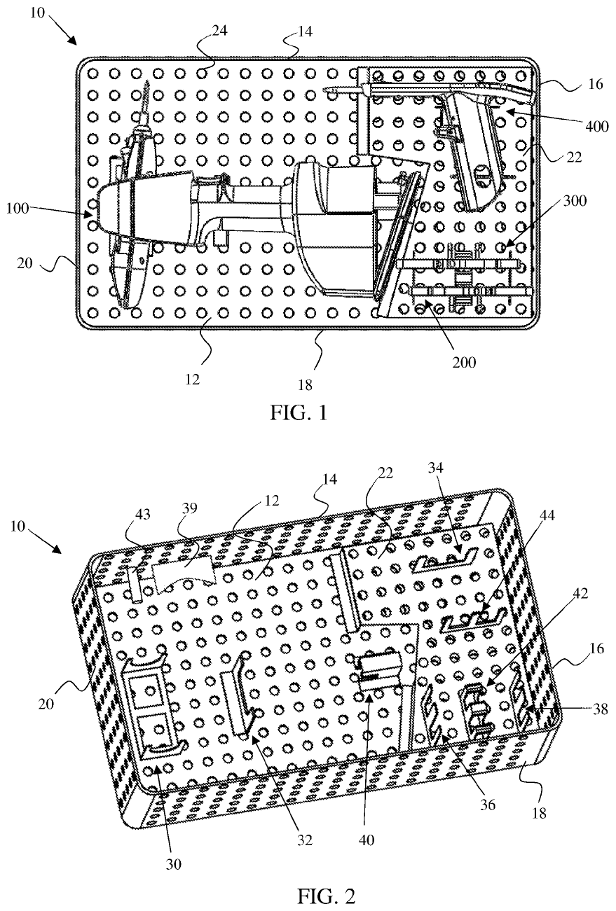

Referring now the drawings, FIG. 1 is a top view of a processing tray 10 with a plurality of devices (100, 200a, 200b, 300) positioned in predetermined positions therein. The tray 10 includes a base 12. In some inventive embodiments, the base is connected to side walls (14, 16, 18, 20). While the inclusion of side walls provides a level of protection and retention for devices, it is noted that a substantially planar tray is well suited for efficient space filling placement in racks within a processing chamber. While the tray 10 is depicted herein as rectilinear in shape, it should be appreciated that an inventive tray is readily formed in a variety of shapes that illustratively include circular, polygonal with from 3 to 12 edges, and irregular shapes similar to a desired device loading thereof. A tray 10 is formed from almost any material so long as it is compatible with the processing condition. A tray is readily formed from a material that illustratively include metals (e.g., steel, tungsten carbide), polymers (e.g., synthetic polymers), or glass. In some inventive embodiments, an insert 22 is provided that either rests on the base 12 or mounts to a wall, if present. In some inventive embodiments, the tray includes openings 24. The openings 24 can assume a variety of shapes and if the tray is formed of expanded material, the openings 24 are routinely circular or diamond-shaped.

The tray 10 includes one or more receiving appurtenances (30, 32, 34, 36, 38, 39) and one or more arresting appurtenances (40, 42, 44). A receiving appurtenance such as those best seen in FIG. 2 are positioned on the base 12 or walls, if present, for retaining the device at a predetermined location on the tray 10. As best seen in FIGS. 2, 3, and 12, a receiving appurtenance (30, 32, 34, 36, 38, 39) may be complementary to the device and assume a variety of shapes to create a complementary feature to a contour of a device (100, 200, 300, 400). Receiving appurtenances may have recesses, prongs, rods, an elevated angle iron, or other such features as shown. With regard to receiving appurtenance 39, it is noted that these can also extend from a side wall (14, 16, 18, 20). At least a portion of the arresting appurtenances as shown may contact the subassembly when the subassembly of the object is coupled with the device to prevent receipt of the device from the predetermined location until the subassembly is decoupled or disengaged to a preselected degree. The arresting appurtenances may therefore be positioned on the base 12 or walls, if present (e.g., arresting appurtenance 43 projects from wall 14), and positioned with respect to the receiving appurtenances to prevent receipt of the device at the predetermined location if a subassembly is not removed from the device. An inventive arresting appurtenance may be in the shape of a hook, an inverted ‘L’, prongs, rods, an elevated angle iron, or other such features as shown, or have a projection member 46 (as shown in FIG. 5) for preventing receipt of an object from which the subassembly has not been displaced from the device. An inventive arresting appurtenance may further include feet 48 to facilitate the adherence to of the tray 10, as best seen in FIG. 5. It is appreciated that appurtenances are readily formed of any of the materials from which the tray is formed.

As will be seen in the following drawings, the exemplary removal of a subassembly (not shown) and retention of a device door in a specific position are assured by the present invention. In some inventive embodiments, a locking feature 41 (e.g., cover, tab, strap, clamp, clip, latch, clasp) is provided to retain a device in the predetermined location. The locking feature 41 may be a separate component of the tray 10, or a portion of the locking feature 41 may be coupled to a receiving appurtenance (30, 32, 34, 36, 38, 39), an arresting appurtenance (40, 42, 44), the base 12, or side walls (14, 16, 18, 20) of the tray 10.

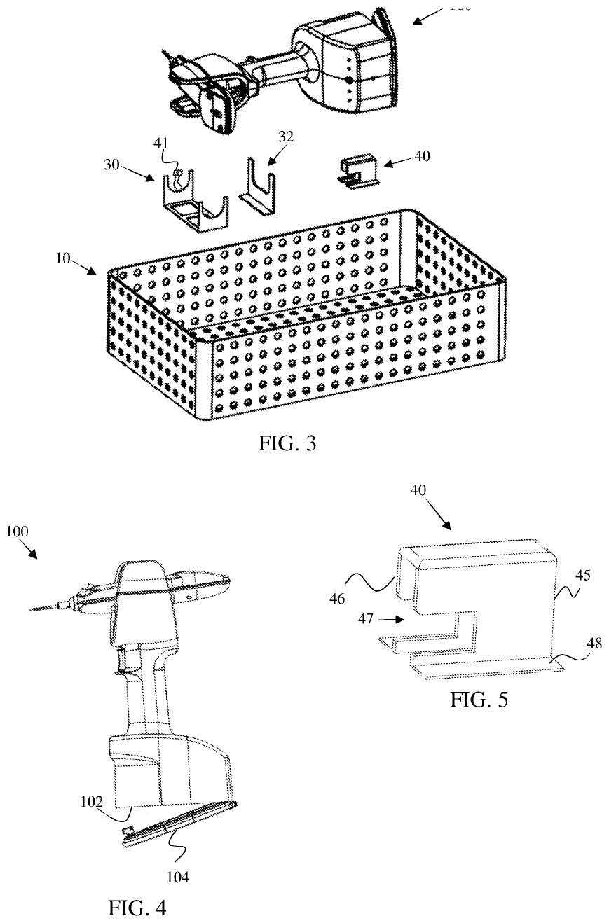

A tray 10 is shown in FIG. 3, 6A, and 6B having receiving appurtenances (30, 32) and an arresting appurtenance 40 for receiving a hand-held robotic device 100. The receiving appurtenances (30, 32) include features to receive and maintain the hand-held robotic device 100 at a predetermined location on the tray 10. The features of the receiving appurtenances (30, 32) may include various projections, prongs, or concavities that cradle a portion of the hand-held robotic device 100. The arresting appurtenance 40 is configured to fit between a device subassembly holder 102 and a door 104 of the hand-held robotic device 100 to ensure any sterilization incompatible subassemblies housed in the device subassembly holder 102 has been removed from the hand-held robotic device 100. If any subassembly remains in the hand-held robotic device 100, then the arresting appurtenance 40 prevents the hand-held robotic device from being received at the predetermined location on the tray 10. As best seen in FIGS. 4 and 5, the arresting appurtenance 40 also functions to assure that the hinged door 104 is retained in an open position to promote process gas or liquid infiltration into the cavity of holder 102. Details of the hand-held robotic device 100 are further described in U.S. patent application Ser. No. 15/778,811 assigned to the assignee of the present application, the contents of which are hereby incorporated by reference. Subassemblies associated with the hand-held robotic device 100 illustratively include a circuit board, micro-processor, data transmitter, data receiver, resistors, capacitors, batteries, and combinations thereof.

FIG. 5 is a perspective view of an arresting appurtenance 40 for preventing receipt of the hand-held robotic device 100 if one or more subassemblies are not removed from the device subassembly holder 102 of the hand-held robotic device 100. The arresting appurtenance 40 may include a body 45, a projection member 46 projecting from the body 45, and feet 48, where a slot 47 is formed between the projection member 46 and the feet 48.

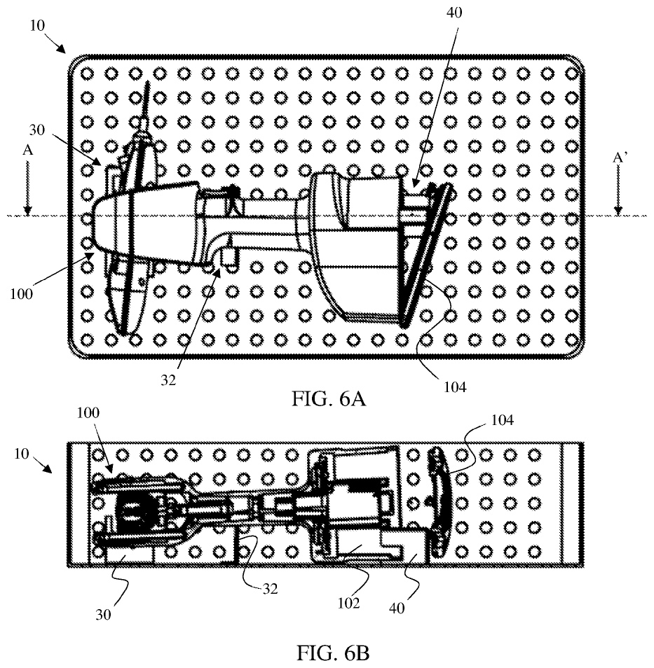

FIG. 6B is a cross-sectional view of FIG. 6A along the dashed line A-A′ showing the arresting appurtenance 40 inside the device component holder 102 to prevent receipt of the hand-held robotic device 100 if a subassembly remains therein. Here, the subassembly has been removed allowing the arresting appurtenance 40 to fit inside the device subassembly holder 102. A wall of the device subassembly holder 102 may be positioned into the slot 47 of the arresting appurtenance 40 where the projection member 46 fits inside the device subassembly holder 102. If a subassembly is not removed from the subassembly holder 102, then the end of the projection member 46 will make contact with the subassembly thereby preventing the wall of the subassembly holder 102 from entering into the slot 47 and receipt of the hand-held robotic device 100 at the predetermined location on the tray 10. Thus, the present invention requires the user to open the door 104 and check for any subassemblies in the device component holder 102 prior to placing the hand-held robotic device 100 on the tray 10.

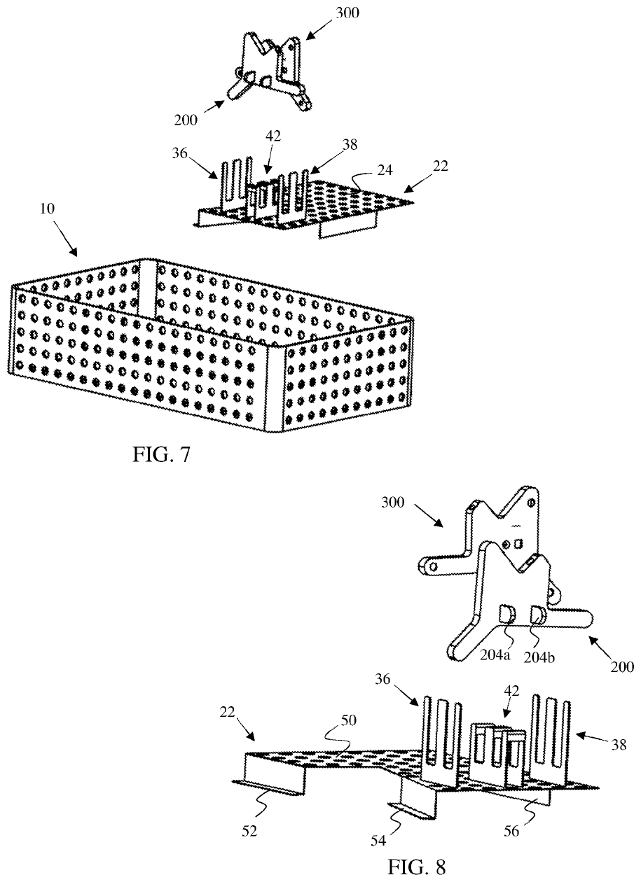

Referring now to FIGS. 7-11, the placement of tracking array devices (200, 300) in an inventive tray 10 are detailed in which like-reference numerals have meaning ascribed thereto with regard to previously described drawings. The insert 22, receiving appurtenances (36, 38), and the arresting appurtenance 42 as shown in exploded view in FIGS. 7 and 8. The insert 22 is located inside the sterilization tray 10 and may provide additional room for devices in the sterilization tray 10, improve fluid flow during sterilization, and/or improves the organizational arrangement of all the devices in the sterilization tray 10. The insert 22 may be removable or fixed inside the tray 10. The insert 22 includes a surface 50. In some inventive embodiments the insert 22 has openings 24. In still other embodiments, the insert 22 includes one or more feet (52, 54, 56) to facilitate the assembly of the insert 22 on the tray 10. One or more receiving appurtenances and/or arresting appurtenances may be assembled to a surface of the insert 22. FIG. 8 depicts the receiving appurtenances (36, 38) and the arresting appurtenance 42 assembled to the insert 22 for receiving two tracking array devices (200, 300) on the tray 10. The receiving appurtenances (36, 38) in some inventive embodiments may include projections and slots wherein a portion of the tracking array devices (200, 300) slide into the slots of the receiving components (36, 38).

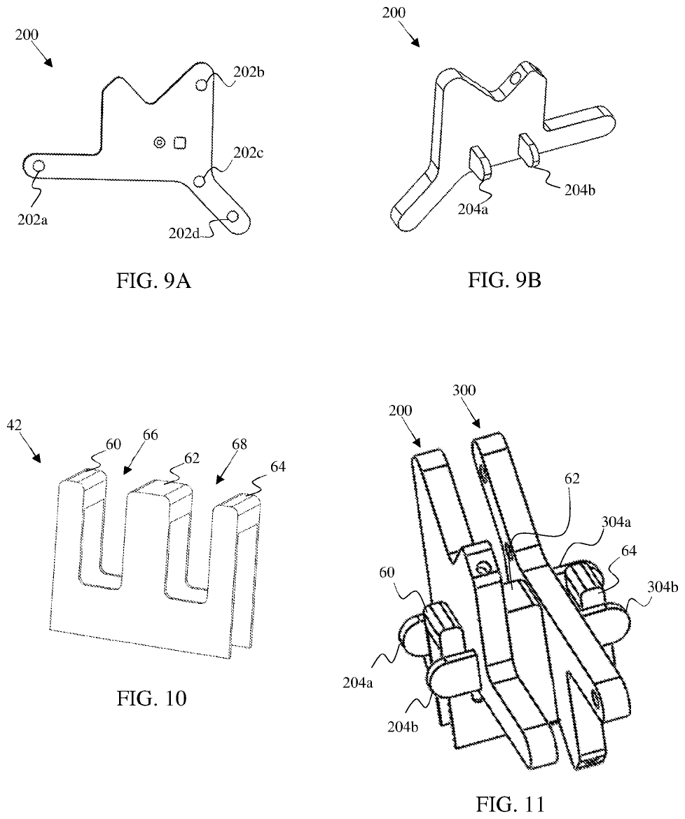

FIGS. 9A and 9B depict the first tracking array device 200, where FIG. 9A is a front view of the tracking array device 200 and FIG. 9B is a rear view of the tracking array device 200. The front side of the tracking array device 200 includes a plurality of fiducial markers (202a, 202b, 202c, 202d). The fiducial markers (202a, 202b, 202c, 202d) each independently illustratively include light emitting diodes (LEDs), or another light source, and typically require a power source. The tracking array device 200 has a pair of battery terminals (204a, 204b). A battery (not shown) is a subassembly that may be incompatible to a sterilization process. Batteries operative herein illustratively include 3 volt, AA, AAA, button-formats, or combinations thereof and typically fit between the battery terminals (204a, 204b) to provide power to the fiducial markers. The arresting appurtenance 42 prevent receipt of the tracking array devices (200, 300) on the tray 10 or insert 22 if a battery subassembly resides between the battery terminals (204, 204b) of either tracking array device (200, 300). The arresting appurtenance 42 includes three prongs (60, 62, 64) in series with a first slot 66 between a first prong 60 and a second prong 64, and a second slot 68 between the second prong 64 and a third prong 64. A first tracking array device 200 slides in the first slot 66 and a second tracking array device 300 slides in the second slot 68. The first tracking array device 200 and second tracking array device 300 are placed in the first slot 66 and second slot 68, respectively, of the arresting appurtenance 42, as best seen in FIG. 11. When in the slots (66, 68), the first prong 60 fits between the battery terminals (204a, 204b) of the first tracking array device 200 and the third prong 64 fits between the battery terminals (304a, 304b) of the second tracking array device 300 thus preventing receipt of either tracking array devices (200, 300) if a battery subassembly is present between the battery terminals (204a & 204b or 304a & 304b) of either tracking array device (200, 300).

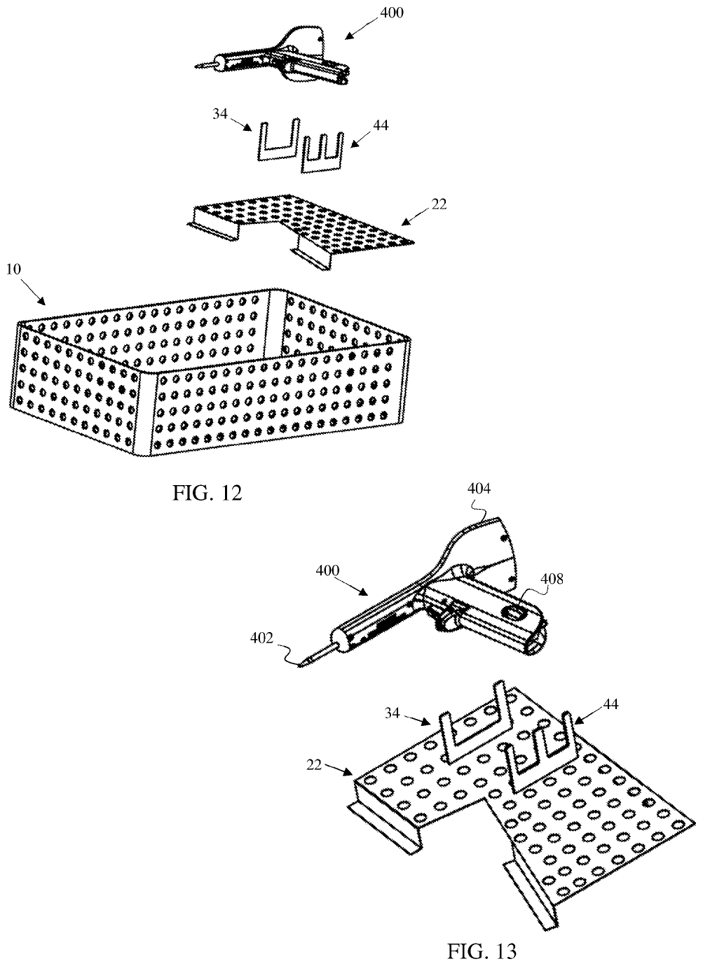

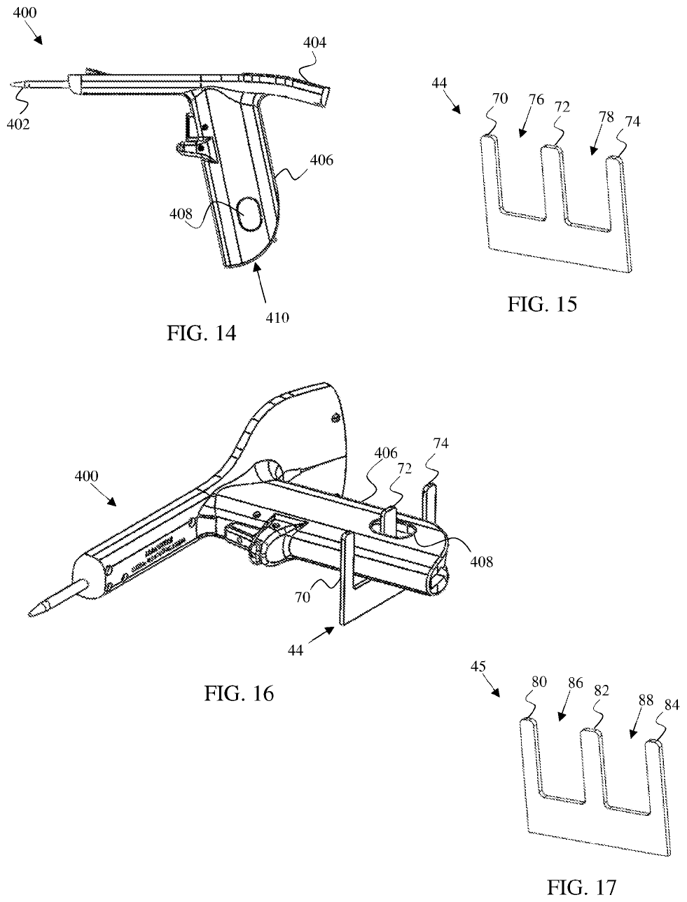

Referring now to FIGS. 12-16, the placement of digitizer device 400 in an inventive tray 10 is detailed in which like-reference numerals have meaning ascribed thereto with regard to previously described drawings. The digitizer device 400 has a probe 402, a digitizer tracking array 404, a digitizer handle 406, and an aperture 408 in the digitizer handle 406. A subassembly containing electrical components for the digitizer device 400 are removably inserted into the digitizer handle 406 via the route shown by arrow 410 in FIG. 14. The arresting appurtenance 44 for the digitizer device 400 includes prongs (70, 72, 74) and slots (76, 78), where a first slot 76 is located between a first prong 70 and a middle prong 72, and a second slot 78 is located between the middle prong 72 and a third prong 74. The digitizer device 400 is received on the arresting appurtenance 44. The middle prong 72 of the arresting appurtenance 44 fits in the aperture 408 of the digitizer handle 406, while portions of the digitizer handle 406 on either side of the hole 408 fit in the first slot 76 and second slot 78 of the arresting appurtenance 44, respectively. Therefore, the middle prong 72 prevents receipt of the digitizer device 400 on the tray 10 or insert 22 if a subassembly resides within the digitizer handle 406 to block the through hole 408.

FIG. 17 depicts an embodiment of an integrated receiving and arresting appurtenance (RAA) 45 having both receiving and arresting appurtenance features and function. RAA 45 includes receiving appurtenances in the form of a first prong 80, a second prong 84, a first slot 86, and a second slot 88, and an arresting appurtenance in the form of a middle prong 82. RAA 45 functions in the same manner as arresting appurtenance 44 for the digitizer device 400.

To process the devices, the tray 10 and devices supported thereon with resort to inventive appurtenances are placed in an appropriate processing chamber and exposed to the desired processing conditions. In the context of sterilization processing, the loaded tray is exposed to conditions that illustratively include ethylene oxide (EtO), high temperatures, steam, chlorine dioxide, actinic radiation, or combinations thereof to affect sterilization. The tray is then removed and the subassembly that did not undergo processing in the chamber is reattached to the device or an new subassembly, perhaps sterilized by another method is coupled to the device.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A processing tray for a device having a subassembly, comprising:
a base;
one or more receiving appurtenances on said base or an insert supported by said base, said one or more receiving appurtenances for retaining the device at a predetermined location; and
one or more arresting appurtenances adapted to prevent receipt of the device at the predetermined location when the subassembly is coupled with the device.

2. The tray of claim 1 wherein the one or more arresting appurtenances are adapted to contact the subassembly when the subassembly is coupled with the device.

3. The tray of claim 1 further comprising walls extending upward from said base.

4. The tray of claim 1 further comprising a plurality of openings in said base.

5. The tray of claim 1 further comprising a wall appurtenance mounted to one of said walls.

6. The tray of claim 1 wherein said base is rectilinear.

7. The tray of claim 1 wherein one of said one or more receiving appurtenances defines a concave cradle on which the device rests.

8. The tray of claim 1 wherein one of said one or more arresting appurtenances defines a prong, hook, or inverted “L” shape adapted to insert into, or around the device only when the subassembly is decoupled from the device.

9. The tray of claim 1 wherein one of said one or more arresting appurtenances defines an angle iron adapted to insert into, or around the device only when the subassembly is decoupled from the device.

10. The tray of claim 2 wherein one of said one or more arresting appurtenances is positioned on at least one of said walls.

11. The tray of claim 1 wherein the subassembly comprises at least one of electronics or a processing condition incompatible material.

12. The tray of claim 11 wherein the electronics comprises at least one of a battery cell, processor, circuit board, transmitter, receiver, capacitor, resistor, or electronic circuitry.

13. The tray of claim 1 wherein said one or more receiving appurtenances, said one or more arresting appurtenances, or a combination thereof further comprising a selective locking feature to retain the device in contact therewith.

14. The tray of claim 1 wherein one of said one or more receiving appurtenances and one of said one or more arresting appurtenances are integrated into a unitary piece.

15. The tray of claim 1 wherein the device is a medical device.

16. The tray of claim 15 wherein the medical device is at least one of a hand-held robotic device, a digitizer, or a tracking array device.

17. A method for processing a device comprising:
decoupling a subassembly from an object to yield the device to be processed;
placing the device in contact with at least a portion of the one or more receiving appurtenances for retaining the device at a predetermined location, wherein one or more arresting appurtenances are positioned to prevent receipt of the device at the predetermined location if the subassembly were coupled to the device; and
exposing the device to a processing condition.

18. The method of claim 17 wherein the object is a medical device.

19. (canceled)

20. The method of claim 17 wherein the subassembly comprises at least one of a battery, a processor, a memory, a peripheral electronic device connection, an electrical connection, a circuit board, a transmitter, a modem, a capacitor, an electrical motor, or a combination thereof.

21. The method of claim 17 wherein the process condition is sterilization.

22. (canceled)
